(19) United States
(12) Patent Application Publication
(10) Pub. No.: US 2022/0249571 A1
(43) Pub. Date: Aug. 11, 2022

(54) NEOINTIMA FORMATION USING MESENCHYMAL STEM CELLS OR DERIVATIVES THEREOF

(71) Applicant: FLUID BIOTECH INC, Calgary (CA)

(72) Inventors: ALIM P. MITHA, Calgary (CA); KAPILAN PANCHENDRABOSE, Winnipeg (CA); ARINDOM SEN, Calgary (CA)

(73) Assignee: FLUID BIOTECH INC., Calgary (CA)

(21) Appl. No.: 17/665,278

(22) Filed: Feb. 4, 2022

Related U.S. Application Data
(60) Provisional application No. 63/146,464, filed on Feb. 5, 2021.

(51) Int. Cl. A61K 35/28; A61P 9/00

(52) U.S. Cl. CPC A61K 35/28; A61P 9/00

(57) ABSTRACT

This disclosure relates to methods for treating vascular conditions using mesenchymal stem cells or a derivate thereof. In particular, this disclosure relates to methods for facilitating neointima formation over an endovascular device or vascular graft through administration of mesenchymal stems cells or a derivate thereof.

### CROSS REFERENCE TO RELATED APPLICATIONS

This application claims the benefit of priority to U.S. Provisional Patent Application No. 63/146,464, filed on Feb. 5, 2021, the disclosure of which is hereby incorporated by reference in its entirety for all purposes.

### BACKGROUND OF THE INVENTION

### 1. Field of Invention

This disclosure relates to methods for treating vascular conditions using mesenchymal stem cells or a derivate thereof. In particular, this disclosure relates to methods for facilitating neointima formation over an endovascular device or vascular graft through administration of mesenchymal stems cells or a derivate thereof.

### 2. Description of Related Art

There is an abundance of medical devices that are known in the art, which are implanted into blood vessels in the body to treat various pathologies. For example, in order to treat an aneurysm within a patient without the need to surgically enter the aneurysm, a flow-diverting scaffold, such as a stent, may be inserted to span the neck of an aneurysm. The stent diverts flow past the aneurysm and thus allows it to heal. However, in the year following flow-diversion, aneurysm persistence rates are typically in the range of 20-45%.

Further, in order to treat restricted flow with a blood vessel due to atherosclerosis, a balloon may be inserted into the vessel in order to force expansion of the blood vessel, followed by the insertion of a stent to ensure the vessel remains open. Such stents may be manufactured from metal mesh, fabric or silicone for example.

In another example, a diseased blood vessel may by bypassed by vascular graft in order to direct blood flow from one area to another. The vascular graft may be derived from the vein of the patient or a donor or a synthetic material such as polytetrafluoroethylene (Teflon™) or polyethylene terephthalate (Dacron®).

However, such procedures can cause complications such as acute and subacute thrombosis which may lead to thromboembolism. This is thought to arise from an immune system response to the foreign material placed in the blood vessel, or from injury caused to adjacent blood vessels during implantation. The immune system response can lead to narrowing of the blood vessel, known as restenosis. As such, patients are required to remain on anti-platelet or anticoagulant medications long-term to reduce the risk of thromboembolism.

It is believed that the risk of thrombus development is significantly reduced after formation of a neointimal layer over the device. The neointimal layer, made of endothelial and other contributing cells to the vessel wall is believed to prevent direct contact of the blood with the device, thus reducing the risk of thrombus development.

Accordingly, there is a need for a method to facilitate neointima formation over an implantable device in order to eliminate or reduce the risk of thrombosis.

### SUMMARY OF THE INVENTION

Aspects of the disclosure relate to a method for facilitating neointima formation over an endovascular device deployed within a vessel of a subject. The method comprises administering an agent to the subject, wherein the agent is mesenchymal stem cells or a derivative thereof.

In various embodiments, the agent is administered before deployment of the endovascular device.

In various embodiments, the agent is administered at the same time as deployment of the endovascular device. In various embodiments, wherein the agent is administered after deployment of the endovascular device. In various embodiments, wherein the agent is administered intra-arterially. In various embodiments, the agent is administered intravenously. In various embodiments, the agent is administered upstream of the endovascular device. In various embodiments, the agent is administered proximal to the endovascular device. In various embodiments, the endovascular device is a flow-diverting scaffold. In various embodiments, the flow-diverting scaffold is a stent. In various embodiments, the endovascular device is coated with vascular endothelial growth factor.

Aspects of the disclosure relate to a method for facilitating neointima formation over a vascular graft implanted on a vessel of a patient. The method comprises administering an agent to the patient, wherein the agent is mesenchymal stem cells or a derivative thereof. In various embodiments, the agent is administered before deployment of the vascular graft. In various embodiments, the agent is administered at the same time as deployment of the vascular graft. In various embodiments, the agent is administered after deployment of the vascular graft.

Aspects of the disclosure relate to a method healing a vascular tear in a patient, comprising administering an agent to the patient, wherein the agent is mesenchymal stem cells or a derivative thereof. In various embodiments, the vascular tear is an arterial dissection.

Aspects of the disclosure relate to a method for facilitating endothelization over an endovascular device deployed within a vessel of a patient. The method comprises administering an agent to the patient, wherein the agent is mesenchymal stem cells or a derivative thereof.

Aspects of the disclosure relate to use of an agent, wherein the agent is mesenchymal stem cells or a derivative thereof, for facilitating endothelization over an endovascular device deployed within a vessel of a patient. In various embodiments, the agent is for administration before deployment of the endovascular device. In various embodiments, the agent is for administration at the same time as deployment of the endovascular device. In various embodiments, the agent is for administration after deployment of the endovascular device. In various embodiments, the agent is for administration upstream of the endovascular device. In various embodiments, the agent is for administration proximal to the endovascular device. In various embodiments, the endovascular device is a flow-diverting scaffold. In various embodiments, the flow-diverting scaffold is a stent. In various embodiments, the endovascular device is coated with vascular endothelial growth factor.

Aspects of the disclosure relate to a method for reducing aneurysm persistence rates after flow-diversion. The method comprises deploying a flow-diverting scaffold within a vessel of a patient and administering an agent to the patient. The agent is mesenchymal stem cells or a derivative thereof. the agent is administered before deployment of the flow-diverting scaffold. In various embodiments, the agent is administered at the same time as deployment of the flow-diverting scaffold. In various embodiments, the agent is administered after deployment of the flow-diverting scaffold. In various embodiments, the agent is administered upstream of the flow-diverting scaffold. In various embodiments, the agent is administered proximal to the flow-diverting scaffold. In various embodiments, the flow-diverting scaffold is a stent. In various embodiments, the flow-diverting scaffold is coated with vascular endothelial growth factor.

Aspects of the disclosure relate to use of an agent, wherein the agent is mesenchymal stem cells or a derivative thereof, for reducing aneurysm persistence rates after flow-diversion. The agent facilitates endothelization over a flow-diverting scaffold deployed within a vessel of a patient. In various embodiments, the agent is for administration before deployment of the flow-diverting scaffold. In various embodiments, the agent is for administration at the same time as deployment of the flow-diverting scaffold. In various embodiments, the agent is for administration after deployment of the flow-diverting scaffold. In various embodiments, the agent is for administration upstream of the flow-diverting scaffold. In various embodiments, the agent is for administration proximal to the flow-diverting scaffold. In various embodiments, the flow-diverting scaffold is a stent. In various embodiments, the flow-diverting scaffold is coated with vascular endothelial growth factor.

Aspects of the disclosure relate to use of an agent, wherein the agent is mesenchymal stem cells or a derivative thereof, for facilitating neointima formation over an endovascular device deployed within a vessel of a patient. In various embodiments, the agent is for administration before deployment of the endovascular device. In various embodiments, the agent is for administration at the same time as deployment of the endovascular device. In various embodiments, the agent is for administration after deployment of the endovascular device.

Aspects of the disclosure relate to use of an agent, wherein the agent is mesenchymal stem cells or a derivative thereof, for facilitating neointima formation over a vascular graft deployed on a vessel of a patient. In various embodiments, the agent is for administration before deployment of the vascular graft. In various embodiments, the agent is for administration at the same time as deployment of the vascular graft. In various embodiments, the agent is for administration after deployment of the vascular graft.

Aspects of the disclosure relate to use of an agent, wherein the agent is mesenchymal stem cells or a derivative thereof, for healing a vascular tear in a patient. In various embodiments, the vascular tear is an arterial dissection.

Aspects of the disclosure relate to use of an endovascular device in combination with an agent, wherein the agent is mesenchymal stem cells or a derivative thereof, for treating a pathology in a vessel of a patient. In various embodiments, the pathology is an aneurysm, a cancer, an infection, coronary heart disease, carotid artery atherosclerotic disease, or intracranial atherosclerosis.

Aspects of the disclosure relate to use of an agent, wherein the agent is mesenchymal stem cells or a derivative thereof, for healing a vascular tear in a patient.

In various embodiments the methods and uses summarized above, the agent is administered intra-arterially. In various embodiments, the agent is administered intravenously. In various embodiments, the agent is administered upstream of the endovascular device. In various embodiments, the agent is administered proximal to the endovascular device.

In various embodiments the methods and uses summarized above, the vessel is an intracranial blood vessel, an aorta, a carotid artery, a vertebral artery, a renal artery or a femoral artery.

In various embodiments the methods and uses summarized above, the vascular graft is coated with vascular endothelial growth factor.

In various embodiments the methods and uses summarized above, about 4×10⁶ mesenchymal stem cells are administered to the patient.

In various embodiments of the methods and uses summarized above, the derivative is microvesicles.

In various embodiments of the methods and uses summarized above, the agent is from an autologous source. In various embodiments, the agent is from an allogenic source.

Aspects of the disclosure relate to a kit for facilitating neointima formation over an endovascular device, the kit comprising an endovascular device and an agent, wherein the agent is mesenchymal stem cells or a derivative thereof.

### BRIEF DESCRIPTION OF THE DRAWINGS

In drawings which illustrate embodiments of the invention,

FIG. 1 depicts neointimal area results from Optical Coherence Tomography at three days;

FIG. 2 depicts neointimal ratio results from Optical Coherence Tomography at three days;

FIG. 3 depicts minimum neointimal thickness results from Optical Coherence Tomography at three days;

FIG. 4 depicts maximum neointimal thickness results from Optical Coherence Tomography at three days;

FIG. 5 depicts stent strut coverage ratio results from Optical Coherence Tomography at three days;

FIG. 6 depicts Scanning Electron Microscopy images at three days for the Day 3 control (left image), Day 3 MSC (center image) and Day 7 Control (right image) groups, where white dashed lines illustrate stent strut outline, scale bar=500 μm;

FIG. 7 depicts mean Scanning Electron Microscopy score at three days.

### Definitions

“Pathology” as used herein refers to the structural and functional deviations from the normal that constitutes or characterizes a disease, condition, or disorder.

“Comprising” as used herein means “including, but not limited to”.

“Consisting” as used herein means “including and limited to”.

“Drug” or “therapeutic agent” as used herein can refer to any of a variety of drugs, pharmaceutical compounds, other bioactive agent that can be used as active agents to prevent or treat a disease.

“Wall” as used herein refers to tissue that forms a tubular structure of a mammalian body including, but not limited to, a blood vessel wall, a ureter wall, a urethra wall, a bile duct wall.

“Endovascular device” as used herein refers to a prosthesis that can be implanted within a body lumen or body conduit. In various embodiments, the endovascular device is a stent.

“Flow-diversion” as used herein refers to diversion of bodily fluid flow away from a pathology.

“Neointima” as used herein refers to a new or thickened layer of intima that that forms within tubular structures

“Body Lumen” as used herein refers to the cavity defined by a tubular structure of a mammalian body including, but not limited to, a blood vessel, a ureter, a urethra, a bile duct.

“Vessel” as used herein refers to a tubular structure of a mammalian body including, but not limited to, a blood vessel, a ureter, a urethra, a bile duct.

“Blood Vessel” as used herein refers to a tubular structure carrying blood, and substances carried by the blood such as nutrients and dissolved gases, throughout a mammalian body.

“Agent” as used herein refers to mesenchymal stem cells (MSCs) or a derivative thereof.

### DETAILED DESCRIPTION

This disclosure generally relates to methods and uses for mesenchymal stem cells or derivatives thereof for forming neointima within a vessel of a patient.

Mesenchymal stem cells (MSCs), also known as mesenchymal stromal cells and medicinal signalling cells, are multipotent stem cells that can differentiate into a variety of tissue types such as bone, cartilage, muscle, fat cells and connective tissue. MSCs may be isolated, purified and cultured from multiple tissue sources such as bone marrow, adipose tissue, blood and umbilical cord tissue, according to methods used by a skilled person. MSCs may be cultured without differentiation or may be induced to differentiate into a selected mesenchymal tissue.

The present inventors have demonstrated that administration of MSCs to a subject facilitates formation of neointima over an endovascular device deployed within the subject. An endovascular device was deployed within the artery of a mammalian subject. MSCs were then administered to the subject, which resulted in an observed increased neointima formation compared to a subject to which MSCs were not administered.

Five days prior to surgical procedures, Male New Zealand White rabbits were started on dual antiplatelet therapy to reduce the risk of thrombosis by orally ingesting Aspirin (10 mg/kg) and clopidogrel (10 mg/kg) medications once per day. The dual antiplatelet regimen was continued every day until euthanasia.

On the day of stent deployment, rabbits were initially anesthetized with an intravenous injection of acepromazine (0.15 mg/kg) into the marginal ear vein. Acepromazine is normally used as a sedative. Rabbits were subsequently intubated with a 3-0 endotracheal tube connected to a ventilator and reached the appropriate level of anesthesia inhaling 5% isofluorane in 100% O₂ at a rate of 1 L/min. Blink reflex, toe pinch and breathing rate was monitored to determine depth of anesthetic induction. Anesthesia was then maintained via 2% isofluorane in 100% O₂ at 1 L/min. Buprenorphine (0.03 mg/kg) was administered subcutaneously for analgesia.

An intravenous line was established in the marginal ear vein to provide maintenance fluids (0.9% saline) at a rate of 4 mL/kg/h for supportive care. Approximately 5 mL of whole blood was collected intra-arterially before the procedure using a butterfly catheter (BD Sad-T-Intima) for aggregometry sampling. The butterfly catheter was also used to administer heparin (100 U/kg) intra-arterially.

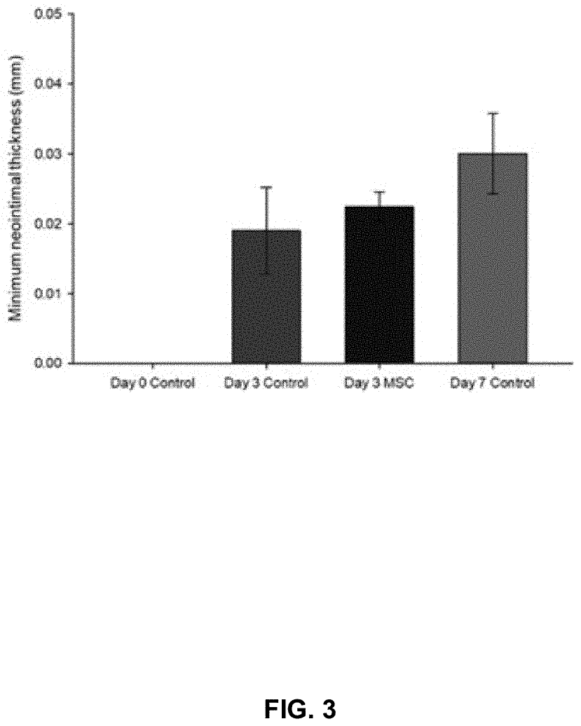

For the surgical procedure, a 3 cm linear incision was made followed by dissection to expose the right femoral artery. The distal end of the femoral artery was ligated using a temporary aneurysm clip and then tied off using 4-0 silk suture 2 cm distal. A 4-French introducer sheath with guide wire (Merit Medical) was inserted into the femoral artery proximal to the ligation (FIG. 3.3). Initial angiography of the infrarenal aorta and its branches was performed with an OEC 9800 C-arm fluoroscopic x-ray unit (General Electric) while administering Optiray® 240 nonionic low-osmolar contrast dye boluses to determine normal patency of aorta and side branches. Contrast dye was rapidly injected while subtracted planar anterior-posterior videos and images were recorded. An 0.021″ inner diameter Prowler Select Plus or 0.025″ Velocity (Penumbra) delivery microcatheter was then inserted into the introducer sheath and navigated to the deployment site within the infrarenal aorta under fluoroscopic guidance.

Solitaire stents (Medtronic, Dublin, Ireland) were compressed into plastic delivery sheaths and detached from their delivery wire just proximal to the most proximal radiopaque marker. The plastic delivery sheath containing the compressed stent was then placed within the Tuohy-Borst hemostatic valve and delivery wire was used to deliver and deploy the stent under fluoroscopic navigation.

A model C7Xr OCT Imaging System (St. Jude Medical) connected to a Dragonfly™ OPTIS™ Imaging Catheter (Abbot) was used to evaluate the neointima formation. Once the catheter was connected to the imaging system, the catheter was purged with contrast dye. The imaging catheter was inserted into the femoral introducer sheath and fluoroscopy was used to guide the catheter so the distal marker was roughly 5 mm beyond the proximal end of the stent. The stented portion of the vessel was then within the image capturing domain of the catheter. For sufficient blood clearance, approximately 10 mL of iodinated contrast dye was administered via the femoral introducer sheath to initiate imaging. Imaging was performed in the automatic setting to self-detect at the point the vessel was free of blood, and set to 20.0 mm per second pull-back speed through 54 mm length to generate 270 frames per pullback.

MSCs were thawed and cultured for seven days in vitro as per standard protocols. On the day of stenting, the MSCs were isolated from culture and then resuspended in 5 mL sterile PBS. The concentrated cell suspension was observed under a light microscope to ensure the absence of cell clumping and any bacterial/fungal contaminations. Once the stents were deployed, the microcatheter was advanced through the stent, but still below the renal arteries of the rabbit in order to obtain an upstream location to potentially maximize effect of the cell injection and to mimic the typical human scenario. Rabbits were administered an intra-arterial injection of 4×10⁶ live MSCs through the microcatheter at a constant rate over one minute. The microcatheter was then flushed with 3 mL saline to maximize cell delivery, then removed from the animal.

After three days, all rabbits were placed under general anaesthetic and a sheath was placed into the femoral artery. Again, OCT images were obtained using the same method described above. OCT images were compared between day 0 and day 3 post-stenting.

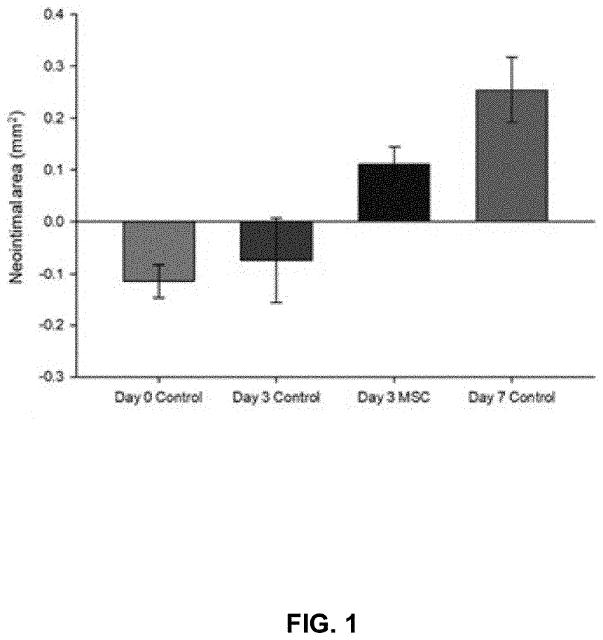

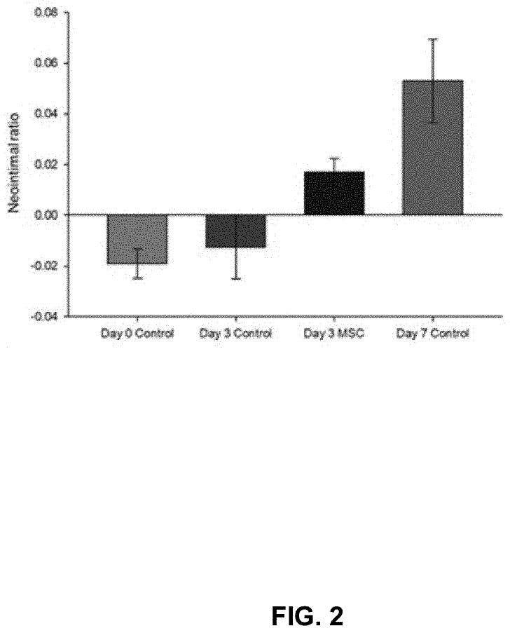

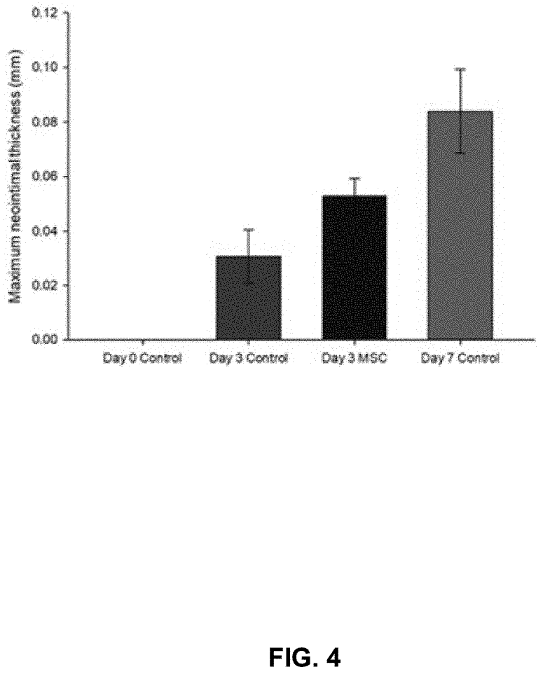

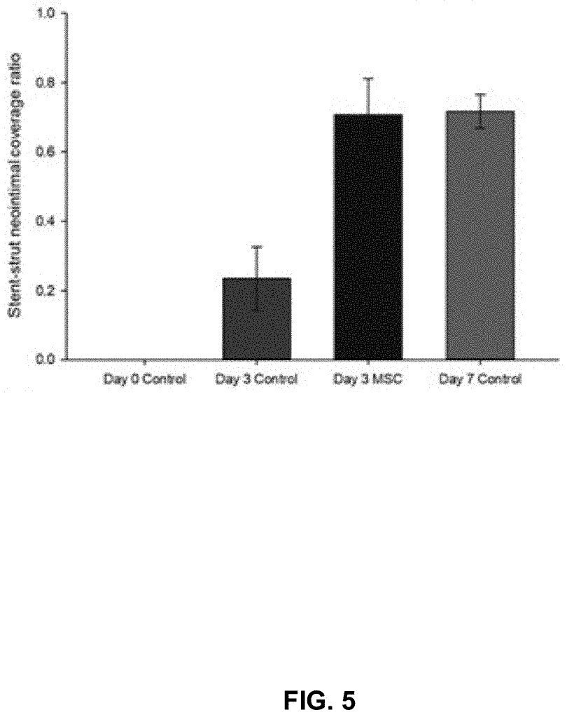

For each specimen, OCT images from the recorded video were systematically saved every 5 mm. Images were analyzed for neointimal area (stent area—lumen area, FIG. 1), neointimal ratio ([stent area—lumen area]/stent area, FIG. 2), minimal (FIG. 3) and maximal (FIG. 4) neointima thicknesses, stent-strut neointima coverage ratio (number of struts covered by a neointimal layer over the total number of struts, FIG. 5). The stent area was defined as the outermost circumferential area limited by the contour of the struts of the stent. The lumen area was defined as the circumferential area defined by the contours of the vessel lumen. The MSC column at 3 days was significantly different compared with the uncoated stent at 3 days in terms of neointimal area, neointimal ratio, and stent strut coverage ratio.

As indicated in FIG. 1, a significant increase in neointimal area was observed for the MSC group at day 3 compared to animals that did not receive MSCs (error bars represent 95% confidence interval).

As indicated in FIG. 2, a significant increase in the neointimal ratio was observed for the MSC group at day 3 compared to animals that did not receive MSCs (error bars represent 95% confidence intervals).

As indicated in FIG. 3, a significant increase in the stent strut coverage ratio was observed for the MSC group at day 3 compared to animals that did not receive MSCs (error bars represent 95% confidence intervals).

As indicated in FIG. 4, an increase in maximum neointimal thickness was observed for the MSC group at day 3 compared to animals that did not receive MSCs (error bars represent 95% confidence intervals).

As indicated in FIG. 5, an increase in minimum neointimal thickness was also observed for the MSC group at day 3 compared to animals that did not receive MSCs (error bars represent 95% confidence intervals).

Scanning Electron Microscopy (SEM) was used to evaluate endothelization over the stent struts in the 3-day control, 3-day MSC and 7-day control groups. After euthanasia, the stent vessel was harvested and irrigated with 10% phosphate-buffered formalin for 10 minutes, then stored in formalin for at least 48 hours. The vessel was cut longitudinally, exposing the lumen and stent.

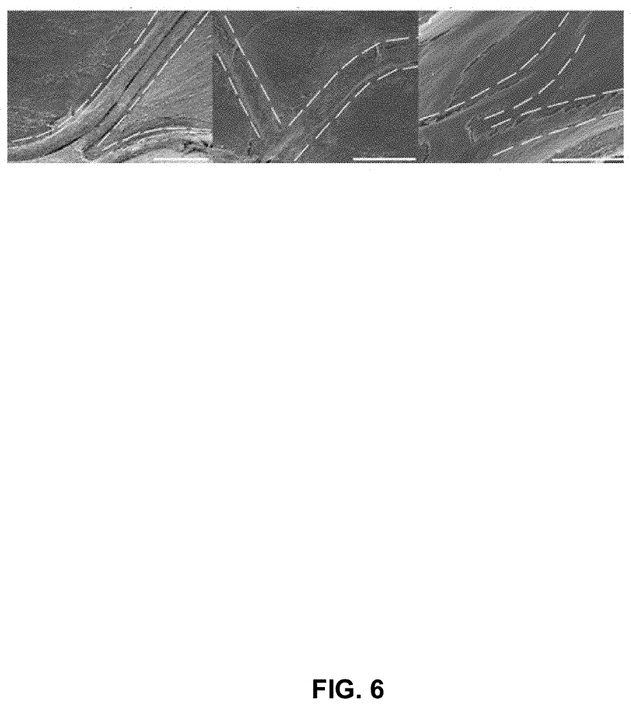

Images were taken en face and a single SEM image at a magnification of approximately ×25 (showing the area of most complete endothelialization for well apposed struts) for each specimen was added to a randomly ordered digital survey (FIG. 6). The survey was sent to three blinded neurointerventionalists (M.E., M.A.A., and A.P.M.) for semiquantitative scoring of endothelialization. The scoring system used was based on a previously described 5-point scale: 0=absent endothelialization, 1=slight endothelialization, 2=moderate endothelialization, 3=marked endo-thelialization, and 4=complete endothelialization.

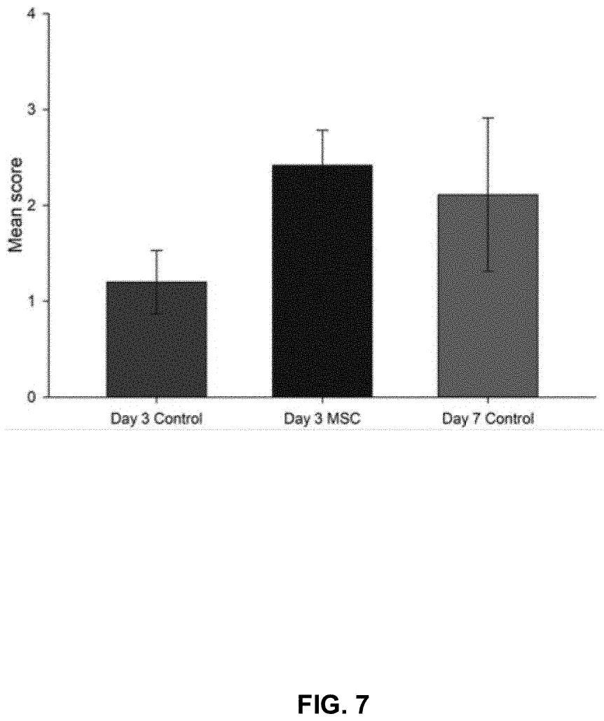

As shown in FIG. 6, the 3-day control showed minimal endothelialization over well-apposed stent struts with several exposed areas. The 3-day MSC and 7-day control groups had generally moderate to marked enodthelialization and more complete embedding of the well-apposed stent struts within the lumen wall. Semiquantitative results for the blinded evaluation of SEM data showed a significant difference in the endothelization score between the 3-day control versus 3-day MSC groups, but nor between any other groups (FIG. 7).

Accordingly, the present results clearly demonstrate that the administration of MSCs to a blood vessel of a subject into which an endovascular device has been deployed facilitates formation of neointima over the endovascular device.

Without being limited to any particular theory, it is believed that, in vivo, the local environment influences differentiation of the mesenchymal stem cells or derivatives thereof into a particular mesenchymal tissue. One such example is the administration of MSCs to a blood vessel that has a region with damaged vascular tissue. It is believed that signals generated by the damaged tissue cause the mesenchymal stem cells to migrate to the damaged vascular tissue where they are induced to differentiate into tissue such as endothelial and smooth muscle cells, facilitating the formation of neointima and endothelial layers at the site of vascular trauma and accelerating the healing process.

While the present example was carried out in rabbits, a skilled person will readily understand wider applications for this methodology for use in treating a wide range of pathologies within vessels of patients. Accordingly, one aspect of the disclosure relates generally to methods for facilitating neointima formation within a vessel of a patient through administration of an agent, wherein the agent is mesenchymal stem cells (MSCs) or a derivative thereof.

One such derivative of MSCs are microvesicles (MVs) of mesenchymal stem cells, which are membrane vesicles secreted from MSCs. MVs may contain cytokines or other signalling factors of MSCs that replicate the same therapeutic benefits, such as facilitating neointima formation.

At least a portion of the agent administered to the patient may reach the deployed endovascular device or vascular graft and facilitate neointima formation.

Furthermore, the methods of the present disclosure are not limited to facilitating neointima formation over an endovascular device or vascular graft. In other embodiments, the agent may be administered to a patient to heal a vascular trauma within a vessel of the patient. In one such embodiment the vascular trauma is a vascular tear and administration of mesenchymal stem cells to the patient may facilitate formation of neointima over the vascular tear. In a specific embodiment, the vascular tear may be an arterial dissection.

As is known to those skilled in the art, in some cases the vascular tear may require surgical treatment that involves deployment of a stent within the damaged vessel, or by deployment of a graft to bypass the damaged region. In these instances, the agent may be administered to the patient in order to facilitate neointima formation over the stent or vascular graft.

In other embodiments, the agent may be administered to a patient in order to treat a pathology within a vessel of a patient, including but not limited to an aneurysm, a cancer, an infection, coronary heart disease, carotid artery atherosclerotic disease, or intracranial atherosclerosis.

In various embodiments, the endovascular device may be a stent deployed within a vessel of the patient in order to treat atherosclerosis or an aneurysm.

For example, the stent may be an aneurysm bridging stent, flow diverting stent, aortic aneurism stent or a stent for peripheral, intracranial or coronary atherosclerotic disease. In an embodiment, the stent is a Solitaire stent manufactured by Medtronic.

In various embodiments, the agent may be administered to a patient to facilitate endothelization of an endovascular device deployed within a vessel of the patient. In an embodiment the endovascular device is a flow diverting scaffold, such as a stent, deployed within a vessel of the patient to treat an aneurysm. The agent may be administered to a patient to reduce aneurysm persistence rates by advantageously facilitating endothelization of the flow diverting scaffold. Through facilitating endothelization of the flow-diverting scaffold the aneurysm may be excluded from circulation, thus reducing the likelihood of aneurysm persistence. Endothelium forms over the flow diverting scaffold, sealing off the aneurysm by diverting blood flow around the site of the aneurysm. This effectively heals the aneurysm because the aneurysm sac is sealed off and the dome of the aneurysm can thrombose.

In embodiments involving a vascular graft, a vascular graft may be deployed in order to bypass a diseased or constricted blood vessel, redirecting blood flow from an area of the vessel with normal blood flow to another area with normal blood flow. In some applications, the vascular graft may be taken from a vein of the patient or a donor. Alternatively, a synthetic vascular graft may be used, made from materials such as polytetrafluoroethylene (Teflon™) or polyethylene terephthalate (Dacron®). As described above for deployment of an endovascular device, the agent administered to the patient may reach the deployed stent and facilitate neointima formation over the vascular graft.

In various embodiments, a section of vessel damaged by, for example, an aortic aneurysm or aortic dissection may be surgically removed, and a vascular graft sutured in place. The agent administered to the patient may reach the vascular graft and promote endothelialisation of the vascular graft.

In various embodiments, the endovascular device may be coated with growth factors that may further stimulate neointima formation over the deployed endovascular device. For example, such growth factors may include vascular endothelial growth factor (VEGF). In other embodiments, the endovascular device could be coated with proteins that promote endothelial cell adhesion such as CD31 and PECAM-1.

In various embodiments, the agent used in the methods described herein will be obtained from the patient being treated (i.e. the agent may have an autologous source). However, the skilled person understands that the agent could be sources from a donor subject (i.e. an allogenic source).

In various embodiments, the vessel may be any body lumen with an endothelial layer, such as a blood vessel, a ureter, a urethra or a bile duct.

In various embodiments, the vessel may be a blood vessel such as an intracranial blood vessel, an aorta, a carotid artery, a vertebral artery, a renal artery or a femoral artery, for example. The skilled person understands that the invention could be used in conjunction with any vessel in which there is a pathology.

While the exemplified embodiment involved administration of MSCs immediately subsequent to (i.e. generally concomitantly with) deployment of the stent, the skilled person understands that, in various embodiments, the may be administered to a patient prior to, during, or after deployment of an endovascular device or vascular graft within a vessel of the patient. The agent may be administered to the patient concomitantly with deployment of the endovascular device or vascular graft. However, a skilled person may recognise that the agent could be administered subsequent to deployment of the endovascular device or vascular graft, for example, 1, 2, 3, or 4 days after deployment. Alternatively, the agent could be administered prior to deployment, for example 1, 2, 3, or 4, days prior to deployment.

In various embodiments, it may be preferable to introduce the agent into the vessel of the patient at a location upstream of the target region being treated (i.e. the pathology or endovascular device as the case may be), such that a proportion of the administered cells preferably migrate to the target region. Alternatively, the agent may be deployed at a position proximal to the target region being treated. However, the skilled person understands that the agent could be administered at a position some distance from the target region if necessary.

In various embodiments, a dose range of about 3×10⁶ to 3×10⁷ mesenchymal stem cells or derivatives thereof may be administered to the patient. The dosage may be administered as an isotonic solution in a suitable liquid such as phosphate buffered saline or other suitable liquids known to those skilled in the art. The solution may be administered to a patient either intra-arterially or intravenously, preferably by injection.

In the exemplified embodiment, a single dose of the agent was administered. However, the skilled person understands that multiple doses of the agent may be administered.

In the exemplified embodiment, the agent was administered intra-arterially. However, the skilled person understands that various embodiments may involve intravenous administration of the agent.

In an embodiment, a cytokine or protein, such as TNF-alpha, IL-1, IL-6, IL-8 may be administered with the agent. The cytokine may promote differentiation of the MSCs into a particular mesenchymal tissue.

In an embodiment, a kit may be provided, including the agent and a stent as described herein. In another embodiment, a kit may be provided, including the agent and a vascular graft as described herein.

Any term or expression not expressly defined herein shall have its commonly accepted definition understood by a person skilled in the art. To the extent that the following description is of a specific embodiment or a particular use of the invention, it is intended to be illustrative only, and not limiting of the invention, which should be given the broadest interpretation consistent with the description as a whole and with the claims.

1. A method for facilitating neointima formation over an endovascular device deployed within a vessel of a patient, comprising administering an agent to the patient, wherein the agent is mesenchymal stem cells or a derivative thereof.

2. (canceled)

3. (canceled)

4. The method of claim 1, wherein the agent is administered after deployment of the endovascular device, before deployment of the endovascular device, or at the same time as deployment of the endovascular device.

5. The method of claim 1, wherein the agent is administered intra-arterially or intravenously.

6-8. (canceled)

9. The method of claim 1, wherein the vessel is an intracranial blood vessel, an aorta, a carotid artery, a vertebral artery, a renal artery or a femoral artery.

10. The method of claim 1, wherein the endovascular device is a stent.

11. The method of claim 1, wherein the endovascular device is coated with vascular endothelial growth factor.

12. The method of claim 1, wherein about 4×10⁶ mesenchymal stem cells are administered to the patient.

13. The method of claim 1, wherein the derivative is microvesicles.

14. The method of claim 1, wherein the agent is from an autologous source or an allogenic source.

15. (canceled)

16. The method of claim 1, wherein a cytokine is administered with the agent.

17. A method for facilitating neointima formation over a vascular graft implanted on a vessel of a patient, comprising administering an agent to the patient, wherein the agent is mesenchymal stem cells or a derivative thereof.

18. (canceled)

19. (canceled)

20. The method of claim 17, wherein the agent is administered after deployment of the vascular graft, before deployment of the vascular graft, or at the same time as deployment of the vascular graft.

21. The method of claim 17, wherein the agent is administered intra-arterially or intravenously.

22-24. (canceled)

25. The method of claim 17, wherein the vessel is an intracranial blood vessel, an aorta, a carotid artery, a vertebral artery, a renal artery or a femoral artery.

26. The method of claim 17, wherein the vascular graft is coated with vascular endothelial growth factor.

27. The method of claim 17, wherein about 4×10⁶ mesenchymal stem cells are administered to the patient.

28-31. (canceled)

32. A method healing a vascular tear in a patient, comprising administering an agent to the patient, wherein the agent is mesenchymal stem cells or a derivative thereof.

33. The method of claim 32, wherein the vascular tear is an arterial dissection.

34. The method of claim 32, wherein the agent is administered intra-arterially or intravenously.

35. (canceled)

36. The method of claim 32, wherein about 4×10⁶ mesenchymal stem cells are administered to the patient.

37-146. (canceled)
